(19) United States
(12) Patent Application Publication
(10) Pub. No.: US 2010/0288283 A1
(43) Pub. Date: Nov. 18, 2010

(54) DYNAMIC ADJUSTMENT OF TUBE COMPENSATION FACTOR BASED ON INTERNAL CHANGES IN BREATHING TUBE

(75) Inventors: Shannon E. Campbell, Boulder, CO (US); Joseph Douglas Vandine, Newark, CA (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/467,113

(22) Filed: May 15, 2009

(51) Int. Cl. A61M 16/04

(52) U.S. Cl. 128/207.14

(57) ABSTRACT

This disclosure describes systems and methods for adjusting a determination of the amount of breathing assistance a patient requires while on a ventilator. In general, in determining the amount of breathing assistance required, the ventilator takes into account an airflow resistance attributable to the tube used to deliver ventilation to the patient's lungs. A tube compensation factor is calculated using a tube compensation algorithm, or similar equation. In particular, the tube compensation factor represents the resistance to airflow attributable to the breathing tube itself based on, inter alia, frictional drag, turbulence, and an internal diameter of the tube. Changes in the tube during ventilation impact the calculation of the breathing assistance required by the patient and are accounted for when compensating for the breathing tube.

A ventilator is a device that mechanically helps patients breathe by replacing some or all of the muscular effort required to inflate and deflate the lungs. Ventilatory assistance is indicated for certain diseases affecting the musculature required for breathing, such as muscular dystrophies, polio, amyotrophic lateral sclerosis (ALS), and Guillain-Barré syndrome Mechanical ventilation may also be required during the sedation associated with surgery and as the result of various injuries, such as high spinal cord injuries and head traumas.

Ventilators may provide assistance according to a variety of methods based on the needs of the patient. These methods include volume-cycled and pressure-cycled methods. Specifically, volume-cycled methods may include among others, Pressure-Regulated Volume Control (PRVC), Volume Ventilation (VV), and Volume Controlled Continuous Mandatory Ventilation (VC-CMV) techniques. Pressure-cycled methods may involve, among others, Assist Control (AC), Synchronized Intermittent Mandatory Ventilation (SIMV), Controlled Mechanical Ventilation (CMV), Pressure Support Ventilation (PSV), Continuous Positive Airway Pressure (CPAP), or Positive End Expiratory Pressure (PEEP) techniques.

Ventilation may be achieved by invasive or non-invasive means. Invasive ventilation utilizes a breathing tube, particularly an endotracheal tube (ET tube) or a tracheostomy tube, inserted into the patient's trachea in order to deliver air to the lungs. Non-invasive ventilation may utilize a mask or other device placed over the patient's nose and mouth.

Ventilators may be configured to determine an amount of breathing assistance a particular patient requires during ventilation. In determining the amount of breathing assistance to deliver, the ventilator will take into account various factors, including the resistance attributable to the equipment that delivers the respiratory gas to the patient's lungs.

This disclosure describes systems and methods for adjusting a determination of the amount of breathing assistance a patient requires while on a ventilator In general, in determining the amount of breathing assistance required, the ventilator takes into account an airflow resistance attributable to the tube used to deliver ventilation to the patient's lungs. The additional resistance is accounted for with a tube compensation factor that is used by the ventilator when determining the amount of breathing assistance required. The tube compensation factor is calculated or otherwise using a tube compensation algorithm, or similar equation, based on information known about the tube being used. In particular, the tube compensation factor represents the resistance to airflow attributable to the breathing tube itself, based on, inter alia, frictional drag, turbulence, and an internal diameter of the tube. Changes in the tube during ventilation impact the calculation of the breathing assistance required by the patient and should be accounted for when compensating for the breathing tube.

There are a variety of reasons that the tube resistance may change during the time a particular patient is connected to the ventilator. Specifically, the tube resistance may increase as a result of a decrease in the internal diameter (ID) of the breathing tube due to a buildup of accretions and/or biofilm formation. Further, depending on the type and amount of this buildup, frictional drag and/or turbulence may hinder airflow within the tube, also increasing the tube resistance.

If a tube compensation algorithm, or similar equation, fails to adequately compensate for increased airflow resistance attributable to the breathing tube, the tube compensation factor can underestimate the amount of breathing assistance required by the patient over time. This underestimation may result in the patient suffering from a lack of adequate oxygen in the short term. Additionally, this underestimation may negatively impact attempts to wean the patient from the ventilator, exposing the patient to numerous risks associated with long-term ventilation and increasing the cost of the patient's treatment.

Embodiments described herein seek to provide methods for dynamically adjusting the tube compensation factor to take into account various internal changes in the breathing tube during ventilation.

In one embodiment, a method for adjusting mechanical ventilation delivered to a patient is disclosed. The method may include determining a first tube compensation factor for an invasive breathing tube through which the patient receives mechanical ventilation and delivering a first appropriate amount of ventilation to the patient based on the first tube compensation factor. The method may also include monitoring elapsed time during ventilation to the patient or internal changes in the breathing tube during ventilation to the patient. The method may then determine a second tube compensation factor and deliver a second appropriate amount of ventilation to the patient based on the second tube compensation factor.

In another embodiment, a medical ventilator is disclosed. The medical ventilator may include one or more sensors adapted to monitor delivery of respiratory gas through a patient circuit and an invasive breathing tube. The medical ventilator may farther include a processor that controls the delivery of respiratory gas through the patient circuit and the invasive breathing tube. The processor may execute a plurality of software modules including a tube compensation factor calculation module that dynamically calculates a tube compensation factor associated with the invasive breathing tube during the delivery of respiratory gas by the medical ventilator. The processor may further execute a respiratory gas delivery module that determines the amount of ventilation to deliver based on a resistance of the patient circuit and the tube compensation factor.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of described technology and are not meant to limit the scope of the invention as claimed in any manner, which scope shall be based on the claims appended hereto,

FIG. 1 is a diagram illustrating a representative ventilator system utilizing an endotracheal tube for air delivery to the patient's lungs.

FIG. 2 is a flow-diagram illustrating methods of adjusting the tube compensation factor as described herein.

FIG. 3 is a block diagram illustrating the disclosed ventilation system.

### DETAILED DESCRIPTION

Although the techniques introduced above and discussed in detail below may be implemented for a variety of medical devices, the present disclosure will discuss the implementation of these techniques for use in a mechanical ventilator system. The reader will understand that the technology described in the context of a ventilator system could be adapted for use with other systems in which variations in a tube resistance to gas flow should be accounted for.

This disclosure describes systems and methods for adjusting the invasive delivery of gas to a patient in response to changes in the condition of the invasive patient interface. The systems and methods presented herein are particularly useful for invasive, longer-term ventilation employing any type of invasive breathing tube.

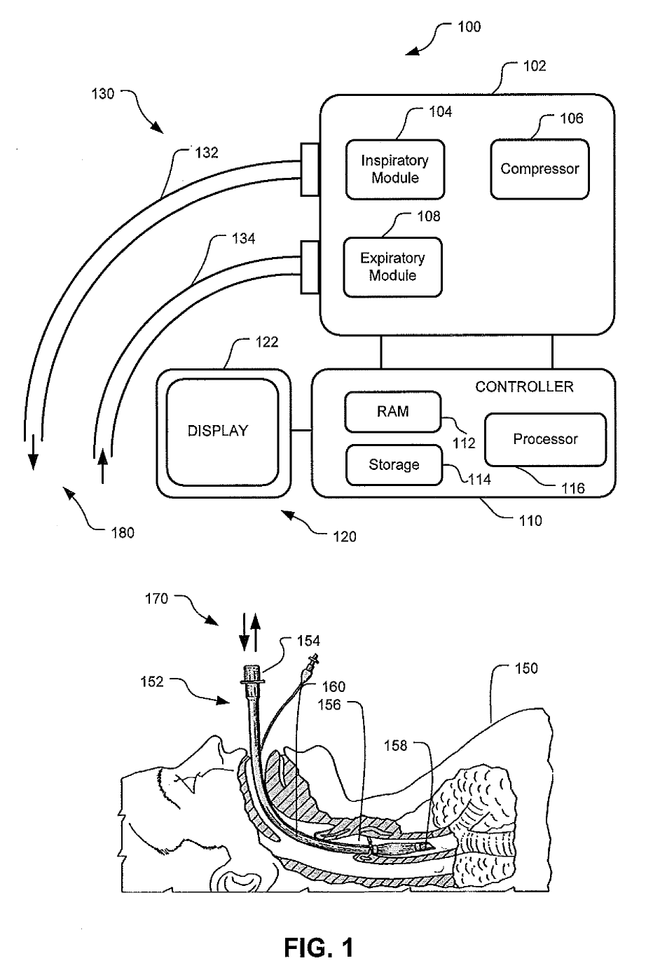

FIG. 1 illustrates an embodiment of a ventilator 100 connected to a human patient 150. Ventilator 100 includes a pneumatic system 102 (also referred to as a pressure generating system 102) for circulating breathing gases to and from patient 150 via the ventilation tubing system 130, which couples the patient to the pneumatic system via an invasive patient interface 152. For the purposes of this disclosure, invasive patient interfaces will be referred to generally as an endotracheal tube (ET tube) although the reader will understand that the technology described herein is equally applicable to any invasive patient interface that utilizes a tube including, tracheostomy tubes, nasopharyngeal airways, and the like as described below.

Airflow is provided between ventilation tubing system 130 and the ET tube 152 and is represented by flow arrows 170 and 180. Ventilation tubing system 130 may be a two-limb (shown) or a one-limb circuit for carrying gas to and from the patient 150. In a two-limb embodiment as shown, a fitting (not shown), typically referred to as a “wye-fitting”, may be provided to couple the patient interface 154 to an inspiratory limb 132 and an expiratory limb 134 of the ventilation tubing system 130.

Pneumatic system 102 may be configured in a variety of ways. In the present example, system 102 includes an expiratory module 108 coupled with the expiratory limb 134 and an inspiratory module 104 coupled with the inspiratory limb 132. Compressor 106 or another source(s) of pressurized gases (e.g., air, oxygen, and/or helium) is coupled with inspiratory module 104 to provide a gas source for ventilatory support via inspiratory limb 132.

The pneumatic system may include a variety of other components, including sources for pressurized air and/or oxygen, mixing modules, valves, sensors, tubing, accumulators, filters, etc. Controller 110 is operatively coupled with pneumatic system 102, signal measurement and acquisition systems, and an operator interface 120 may be provided to enable an operator to interact with the ventilator 100 (e.g., change ventilator settings, select operational modes, view monitored parameters, etc.). Controller 110 may include memory 112, one or more processors 116, storage 114, and/or other components of the type commonly found in command and control computing devices.

The memory 112 is computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. Computer-readable storage media includes volatile and non-volatile, removable and non-removable media implemented in any method or technology for storage of information such as computer-readable instructions, data structures, program modules or other data. Computer-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

As described in more detail below, controller 110 issues commands to pneumatic system 102 in order to control the breathing assistance provided to the patient by the ventilator. The specific commands may be based on inputs received from patient 150, pneumatic system 102 and sensors, operator interface 120 and/or other components of the ventilator. In the depicted example, operator interface includes a display 122 that is touch-sensitive, enabling the display to serve both as an input and output device.

The ET tube 152 is a long, flexible tube that is inserted into the trachea (windpipe) 156 of a patient to ensure that the patient's airway is held open so that air is able to reach the lungs. An ET tube is inserted through the patient's nose or mouth in a process called intubation. A tracheostomy tube (“trach” tube) (not shown) is inserted by way of a tracheostomy (sometimes referred to as a tracheotomy) through the neck directly into the trachea 156 of a patient. Currently, the endotracheal and tracheostomy tubes are regarded as the most reliable available method for protecting a patient's airway during mechanical ventilation.

The present disclosure is particularly applicable to ET tubes 152, which are longer and not as easily cleaned and suctioned as tracheostomy tubes. However, some or all of the various systems and methods may be equally adaptable to a patient ventilation system delivered through a tracheostomy tube.

Endotracheal tubes 152 may be made of any suitable non-toxic, flexible material, for example siliconized polyvinyl chloride (PVC), polyurethane, or other appropriate materials. Many ET tubes also include a cuff portion located near the distal end of the ET tube that prevents air and fluid leakage and promotes proper tube placement. ET tubes are known in the art and the technology described herein is applicable to any ET tube now known or later developed.

In some embodiments, ET tubes 152 employ “smart tube” technologies wherein electronic chips and sensors are provided within the ET tube. Smart tube technologies provide electrical connections (physical or wireless) from the tube to the ventilator or other monitor whereby discrete changes within the tube or at the tube distal end may be detected and communicated to the ventilator or other monitor. Further, each smart ET tube may include a unique identification chip, enabling the ventilator or monitor to detect the type and size of a particular ET tube employed, to detect if and when an ET tube is replaced, and other tube or airflow characteristics.

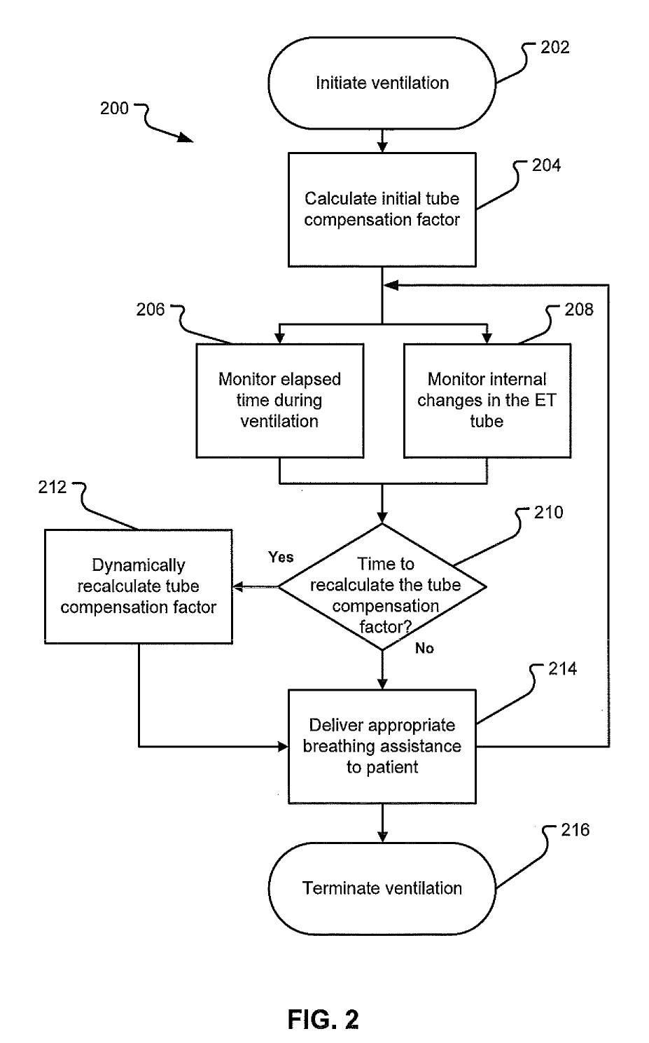

FIG. 2 is a flow-diagram illustrating the processes described herein. At 202, ventilator 100 initiates ventilation to a patient, e.g., patient 150. In order to deliver the appropriate amount of ventilation to patient 150, the ventilator determines an initial tube compensation factor attributable to the ET tube 152 by utilizing a tube compensation algorithm, or similar equation, at 204.

In addition to determining the tube compensation factor, in an embodiment the tube compensation algorithm may also compensate the pressure and flow to be delivered to the patient by the ventilator based on the tube compensation factor. In an alternative embodiment, the tube compensation algorithm may only supply the tube compensation factor to the ventilator's controller, which then performs the compensation when calculating the flow and pressure to be delivered to the patient.

For the purposes of this application, the term “tube compensation factor” is used to generally indicate any value or information usable by the ventilator in determining how much to adjust ventilation in order to compensate for the resistance introduced by the ET tube. Thus, a tube compensation factor may be a resistance change, a pressure drop, a flow impedance or any other parameter. For example, in an embodiment of a ventilator that uses pressure drop to characterize the resistance of the patient circuit and the ET tube, the tube compensation factor may be a resistance value that the ventilator adds to the resistance of the patient circuit before determining the amount of ventilation to provide to the patient.

In an embodiment, the tube compensation algorithm may calculate the initial tube compensation factor taking into account the internal diameter (ID) of the particular ET tube employed during ventilation. This ID may be entered into the ventilator system by a user, or in some smart tube embodiments calculated by or provided to the ventilator. In some embodiments, various other factors, such as ET tube length, surface roughness, etc, are used in calculating the compensation factor. As is known in the art, variations in the ID of a tube exponentially affect the resistance to gas flow through the tube. Thus, even small changes in the ID can affect the delivery of appropriate breathing assistance to a patient. In an alternative embodiment, the tube compensation algorithm may include selecting a predetermined initial tube compensation factor based on the ID, length, model number or other identifier of the ET tube.

The ID of the ET tube 152 may vary with the materials and the manufacturing processes employed. Further, ET tubes are sized such that the different physical attributes of patients are considered. For instance, ET tubes may be sized for infants, children, adult women, adult men, etc. IDs typically fall within the range of 2.0 to 10.0 millimeters and ET tube sizes increase by 0.5 millimeter ID increments.

Previously, the ID for a particular ET tube was utilized as a constant in calculations determining the tube compensation factor. This disclosure proposes that ventilator calculations will more accurately predict the amount of breathing assistance required by a patient if the ID is utilized as a variable that is recalculated periodically in order to accurately track changes in the ID. Specifically, as a result of biofilm growth and/or accretions building up on the inner surfaces of the ET tube, the ET tube's ID may decrease substantially over time, causing an increase in the ET tube's resistance to airflow. This change in resistance results in a change in tube compensation factor that impacts the ventilator calculation of the appropriate amount of breathing assistance to be delivered to the patient.

At 208, the ventilator monitors internal changes in the ET tube, including changes in the ID, surface roughness, frictional drag and/or flow turbulence within the ET tube due to accretions and/or biofilm buildup. Accretions include mucous and moisture, either from the lungs or from the nose and mouth that has leaked into the lung during ventilation. Moisture may collect in droplets or channels, creating an uneven internal tube surface. Further, mucous may adhere to the internal tube surface in uneven mounds as a result of its high glycoprotein content. Biofilm formation, resulting from the activity of bacteria and other microorganisms, exhibits a high carbohydrate composition and may cause uneven granular deposits on the internal walls of the ET tube.

In addition to decreasing the absolute ID of an ET tube, the accretion and/or biofilm buildup in the ET tube may also increase frictional drag and/or air turbulence within the ET tube, further negatively influencing airflow within the tube, and increasing the tube resistance. The increased turbulence is a result of the uneven nature of the deposit buildup attributable to the accretions and/or biofilm. Increased frictional drag is attributable to an increase in surface roughness along the internal surface of the ET tube due to the deposit buildup.

Data suggests that a decrease in the ID due to accretions and/or biofilm buildup is relatively consistent along the interior length of the tube. However, the proximal end of the ET tube 158, in closer proximity to the lungs, may exhibit additional buildup. This is due in part to the fact that the proximal end 158 is not easily suctioned and also to the fact that the oro-pharyngeal bend 160 of the breathing tube encourages the collection of moisture and mucous at the proximal end of the tube.

Significantly, the degree of accretion and/or biofilm buildup is highly patient-specific. In fact, the ID may decrease by a full size within as little as four hours for some patients. Over longer periods, the ID may decrease by as many as three full sizes (approximately 1.5 mm). This is especially serious in light of the exponential impact the internal radius has on the tube compensation factor. In one embodiment, at 206, the ventilator monitors the elapsed time during ventilation. At predetermined increments of time, e.g. four-hour increments, the ventilator may determine that recalculation of the tube compensation factor is necessary at 210.

The patient-specific nature of the accretion and/or bioflim buildup, in terms of both the extent of buildup and of the rate at which buildup occurs, suggests that a standardized method of predicting accretion and/or biofilm buildup over time, as at 206, may not be as accurate as a patient specific one. Thus, at 208, some embodiments of the claimed methods utilize sensors, mathematical flow calculations, or smart tube technologies to monitor internal changes in the ET tube on an individualized patient basis, including changes in the ID, frictional drag and/or flow turbulence within the ET tube.

Specifically, at 208, embodiments of the present disclosure may utilize mathematical means to determine changes in the ET tube. For example, computational fluid dynamics (CFD) may be employed to determine changes in the ID or in the ET tube frictional drag or flow turbulence as compared to baseline calculations.

Embodiments of the present disclosure may also utilize electronic sensors within a “smart” ET tube, as described above, at 208. Smart tube technologies enable the ventilator to detect even discrete changes along the interior of the ET tube.

Embodiments of the present disclosure may also utilize sensors to determine a decrease in the ID due to accretions and/or biofilm buildup at 208. Specifically, one or more sensors may be affixed to the cuff portion of the ET tube or may be imbedded in the plastic tubing itself. For example, a pressure transducer may be attached at the distal end of the ET tube to monitor changes in tube pressure at that location. Alternately, sensors may utilize optical or ultrasound techniques for directly measuring changes in the ID and/or tube airflow. Additionally, computerized axial tomography (CT or CAT) scanning or magnetic resonance imaging (MRI) technologies may be employed at 208 to image and detect internal changes in the ET tube.

At 210, it is determined whether recalculation of the tube compensation factor is necessary. In one embodiment, as described above, after a predetermined amount of time has elapsed, the tube compensation factor may be recalculated at 212 based on a formula that takes into account average changes in the ET tube over time, e.g. four-hour increments. This embodiment may be appropriate where sensing and measuring techniques are unavailable or cost-prohibitive. In another embodiment, detecting specified changes in the pressure and flow response of the system indicative of a change in the resistance of the ET tube may be used to trigger the recalculation at 212.

In an embodiment, when changes in the ET tube have been detected, it is determined that a recalculation of the tube compensation factor is necessary at 210. The tube compensation factor is dynamically recalculated at 212. The recalculation of the tube compensation factor takes into account any decrease in ID over a previous ID measurement. Further, the recalculation adjusts for increases in frictional drag and/or flow turbulence within the ET tube. After dynamic recalculation of the tube compensation factor, the ventilator delivers an appropriate amount of ventilation to patient 150 at 214.

The recalculation at 212 may include comparing the current pressure drop necessary to obtain a certain flow in the ET tube to a previously determined pressure drop (such as the initial pressure drop as determined by the initial tube compensation factor) for the same flow in order to determine the relative change in resistance. This relative change may then be used to calculate a revised tube compensation factor for the ET tube. Alternately, any other suitable method for determining a change in tube resistance, and for revising the tube compensation factor, may be employed.

If it is determined that dynamic recalculation of the tube compensation factor is not necessary at 210, the ventilator proceeds to 214 and delivers an appropriate amount of breathing assistance to patient 150 based on the immediately previous calculation of the tube compensation factor.

Finally, after consistently delivering the appropriate amount of ventilation to patient 150, patients who recover are successfully weaned from the ventilator and ventilation is terminated at 216.

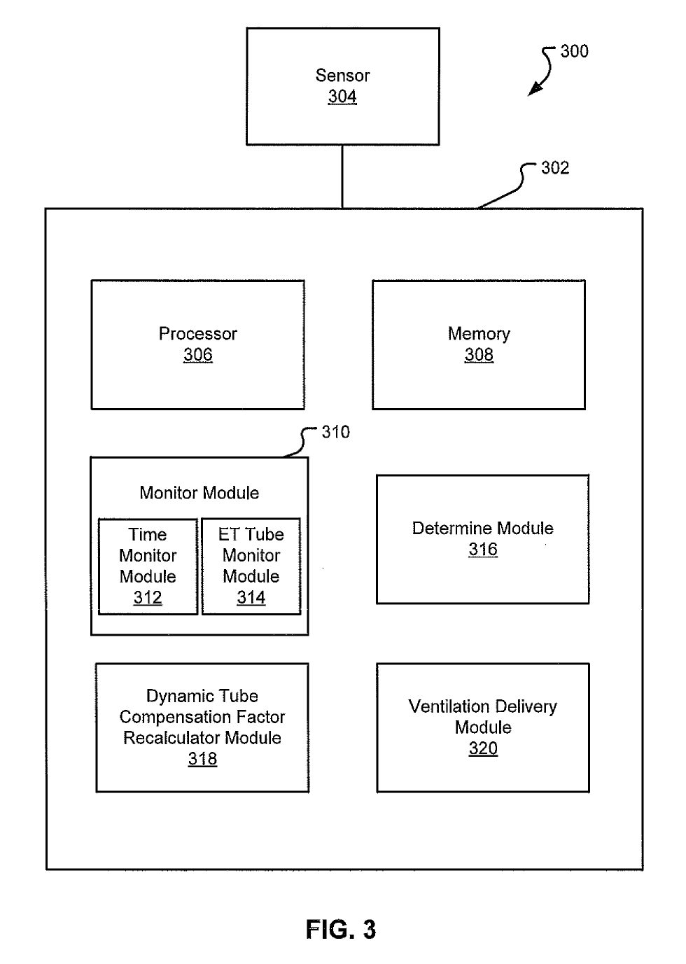

FIG. 3 is a block diagram illustrating the disclosed ventilation system 300. The ventilator 302 includes various modules 310-320, memory 308 and one or more processors 306. Memory 308 is defined as described above for memory 112. Similarly, the one or more processors 306 are defined as described above for the one or more processors 116.

Sensor 304 conducts measurements of internal changes in the ET tube, including changes in one or more of the ID, frictional drag and/or of the flow turbulence within the ET tube. As such, Sensor 304 may include any suitable sensory device, including sensory devices employing optical, ultrasound, or pressure sensitive methods as described above. Sensor 304 may also include any suitable device that, rather than sensing internal changes in the ET tube, uses mathematical means, such as computational fluid dynamics (CFD), to calculate discrete changes within the ET tube. Sensor 304 may also involve CT or MRI tube imaging methods, used to image at least a portion of the ET tube such as the proximal end.

Sensor 304 communicates internal changes in the ET tube to the Monitor Module 310, and specifically to an ET Tube Monitor Module 314. Monitor Module 310 communicates with a Determine Module 316.

In some embodiments, a Time Monitor Module 312, monitors the elapsed time during ventilation of the patient 150. Time Monitor Module 312 communicates with Monitor Module 310, which in turn communicates with Determine Module 316.

Determine Module 316, after receiving information regarding elapsed time and/or information regarding internal changes in the ET tube from Monitor Module 310, determines whether it is necessary to dynamically recalculate the tube compensation factor. When Determine Module 316 determines that it is necessary to dynamically recalculate the tube compensation factor, it initiates a Dynamic Tube Compensation Factor Recalculator Module 318. When Determine Module 316 determines that recalculation of the tube compensation factor is unnecessary, it initiates a Ventilation Delivery Module 320.

Dynamic Tube Compensation Factor Recalculator Module 318 recalculates the tube compensation factor and determines the appropriate breathing assistance needed by patient 150. The tube compensation factor is recalculated based on internal changes in the ET tube, including changes in the ID or changes in internal frictional drag and/or flow turbulence within the ET tube. Upon recalculation of the tube compensation factor by Dynamic Tube Compensation Factor Recalculator Module 318, Ventilation Delivery Module 320 provides the appropriate amount of ventilation to patient 150.

It will be clear that the systems and methods described herein are well adapted to attain the ends and advantages mentioned as well as those inherent therein. Those skilled in the art will recognize that the methods and systems within this specification may be implemented in many manners and as such is not to be limited by the foregoing exemplified embodiments and examples. In other words, functional elements being performed by a single or multiple components, in various combinations of hardware and software, and individual functions can be distributed among software applications at either the client or server level. In this regard, any number of the features of the different embodiments described herein may be combined into one single embodiment and alternate embodiments having fewer than or more than all of the features herein described are possible.

While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

What is claimed is:

1. A method for adjusting mechanical ventilation delivered to a patient, comprising:
determining a first tube compensation factor for an invasive breathing tube through which the patient receives mechanical ventilation;
delivering a first appropriate amount of ventilation to the patient based on the first tube compensation factor;
monitoring at least one of: elapsed time during ventilation to the patient and internal changes in the breathing tube during ventilation to the patient;
determining a second tube compensation factor;
delivering a second appropriate amount of ventilation to the patient based on the second tube compensation factor.

2. The method of claim 1, wherein monitoring internal changes in the breathing tube during ventilation to the patient comprises:
monitoring changes in an internal diameter (ID) of the breathing tube due to accretion buildup within the breathing tube.

3. The method of claim I, wherein monitoring internal changes in the breathing tube during ventilation to the patient comprises:
monitoring changes in an ID of the breathing tube due to biofilm growth within the breathing tube.

4. The method of claim 1, wherein monitoring internal changes in the breathing tube during ventilation to the patient comprises:
monitoring changes in surface roughness within the breathing tube.

5. The method of claim 1, wherein monitoring internal changes in the breathing tube during ventilation to the patient comprises:
monitoring changes in a turbulence within the breathing tube due to at least one of: accretion buildup and biofilm growth.

6. The method of claim 1, wherein monitoring internal changes in the breathing tube during ventilation to the patient comprises:
monitoring changes in the breathing tube using one or more electronic sensors in the tube.

7. The method of claim 1, wherein monitoring internal changes in the breathing tube during ventilation to the patient comprises:
monitoring changes in the breathing tube using a pressure transducer associated with the breathing tube.

8. The method of claim 1, wherein monitoring internal changes in the breathing tube during ventilation to the patient comprises:
monitoring changes in the breathing tube using at least one sensor associated with the breathing tube from the group consisting of: an optical sensor and an ultrasound sensor.

9. The method of claim 1, wherein monitoring internal changes in the breathing tube during ventilation to the patient comprises:
monitoring changes in the breathing tube using computational fluid dynamics calculations.

10. The method of claim 1, wherein determining the second tube compensation factor comprises:
monitoring the elapsed time during ventilation; and
at a desired length of elapsed time of ventilation, setting the second tube compensation factor to a desired value.

11. The method of claim 10, wherein the desired value comprises a first compensation factor for a second endotracheal tube, the second endotracheal tube having an initial internal diameter smaller than the breathing tube.

12. The method of claim 1, wherein calculating a first tube compensation factor for a breathing tube through which the patient receives mechanical ventilation comprises:
calculating a first tube compensation factor for an endotracheal tube.

13. A medical ventilator comprising:
one or more sensors adapted to monitor delivery of respiratory gas through a patient circuit and an invasive breathing tube;
a processor that controls the delivery of respiratory gas through the patient circuit and the invasive breathing tube, the processor executing a plurality of software modules including:
a tube compensation factor calculation module that dynamically calculates a tube compensation factor associated with the invasive breathing tube during the delivery of respiratory gas by the medical ventilator.

14. The medical ventilator of claim 13 further comprising:
a respiratory gas delivery module that determines an amount of ventilation to deliver based on a resistance of the patient circuit and the tube compensation factor; and
wherein the tube compensation factor calculation module is adapted to provide the tube compensation factor to the respiratory gas delivery module.

15. The medical ventilator of claim 13 wherein the tube compensation factor calculation module calculates the tube compensation factor based on data provided by at least one sensor.

16. The medical ventilator of claim 13 wherein the tube compensation factor is a measure of resistance to gas flow of the invasive breathing tube.

17. The medical ventilator of claim 13 wherein the tube compensation factor is a pressure differential.

18. The medical ventilator of claim 13 wherein the invasive breathing tube is one of an endotracheal tube and a tracheostomy tube.

19. The medical ventilator of claim 13 wherein the tube compensation factor calculation module calculates the tube compensation factor based on a duration of ventilation.
